(19) United States
(12) Patent Application Publication
(10) Pub. No.: US 2012/0109129 A1
(43) Pub. Date: May 3, 2012

(54) REPLACEMENT SYSTEM FOR A SURGICAL WIRE

(76) Inventor: Oren S. Bernstein, Portland, OR (US)

(21) Appl. No.: 13/287,955

(22) Filed: Nov. 2, 2011

Related U.S. Application Data
(60) Provisional application No. 61/409,415, filed on Nov. 2, 2010.

(51) Int. Cl. A61B 17/82

(52) U.S. Cl. 606/74

(57) ABSTRACT

System, including methods, apparatus, and kits, for replacing a damaged surgical wire, such as a surgical wire that has broken during or after installation around bone. The system may include a connector with at least one ferrule for attaching a substitute wire to a damaged wire and provides a method of replacing a damaged wire with a substitute wire by using the damaged wire as a leader for travel of the substitute wire around bone.

### CROSS-REFERENCE TO PRIORITY APPLICATION

This application is based upon and claims the benefit under 35 U.S.C. §119(e) of U.S. Provisional Patent Application Ser. No. 61/409,415, filed Nov. 2, 2010, which is incorporated herein by reference in its entirety for all purposes.

### INTRODUCTION

The rib cage, or thoracic cage, is composed of bone and cartilage that surround the chest cavity and organs therein, such as the heart and the lungs. In humans, the rib cage typically consists of 24 ribs, twelve thoracic vertebrae, the sternum (or breastbone), and the costal cartilages. The ribs articulate with the thoracic vertebrae posteriorly and, with the exception of the bottom two pairs of ribs (the floating ribs), are connected to the sternum anteriorly by the costal cartilages.

Major surgery inside the chest cavity, such as open heart surgery, generally requires that the rib cage be opened. The most common procedure for opening the rib cage is for a surgeon to place a longitudinal cut through the entire length of the sternum, from the sternal notch superiorly to the xiphoid process inferiorly, in a procedure called a median sternotomy. Cutting the sternum forms left and right sternal halves. The surgeon then separates the sternal halves by urging them apart from one another, to gain access to the chest cavity. After surgery in the chest cavity, the sternal halves are brought back together and can be secured to one another in bony approximation using wires as sutures.

The surgeon may close the sternum after median sternotomy using a cerclage procedure in which wires as sutures encircle the sternum at intercostal positions along the sternum. A wire suture also may be placed into the sternum, in line with the most superior pair of costal cartilages, to extend through, rather than around, the top region of the sternum (the manubrium).

Each wire may be pre-attached to a curved needle that is used to guide the wire around (or through) each sternal half. Generally, all of the wires are positioned around the sternal halves while the halves are spaced from one another (i.e., while the chest cavity is open), which simplifies wire placement, allows use of a shorter needle, and minimizes the chance of piercing the heart with the needle.

Each wire may be tensioned and secured around the sternum. The most common procedure is to twist the ends of the wire together. With this low-tech approach, twisting the wire ends applies increasing tension to the wire and draws the sternal halves into engagement with one another, to provide secure bony approximation of the sternal halves.

A disadvantage of this low-tech approach for sternal closure is breakage of the wire: twisting the wire ends may fracture the wire. Once a wire breaks, the surgeon may choose to re-open the chest cavity, to allow a replacement wire to be placed around the sternum with less risk of heart puncture. Accordingly, if other wires already have been secured around the sternum, the surgeon removes and replaces these other wires, too. The net result can be more time in the operating room, increased risk to the patient, and frustration for the surgeon.

Therefore, an improved cerclage system is needed for closure of the sternum, particularly a system that permits replacement of a broken wire without re-opening the chest cavity.

### SUMMARY

The present disclosure provides a system, including methods, apparatus, and kits, for replacing a damaged surgical wire, such as a surgical wire that has broken during or after installation around bone. The system may include a connector with at least one ferrule for attaching a substitute wire to a damaged wire and provides a method of replacing a damaged wire with a substitute wire by using the damaged wire as a leader for travel of the substitute wire around bone.

### BRIEF DESCRIPTION OF THE DRAWINGS

FIG. 1 is a generally anterior, fragmentary view of a cut sternum having a broken wire extending around a posterior region of the sternum, with the broken wire attached via a connector (a single ferrule) to a substitute wire of a wire replacement system, in accordance with aspects of the present disclosure.

FIG. 2 is a side view of the ferrule of FIG. 1, taken in the absence of the sternum and the wires before the ferrule has been attached to either wire.

FIG. 3 is a sectional view of the ferrule of FIG. 2, taken generally along line 3-3 of FIG. 2.

FIG. 4 is a side view of another exemplary ferrule for use in the wire replacement system of FIG. 1, in accordance with aspects of the present disclosure.

FIG. 5 is a side view of still another exemplary ferrule for use in the wire replacement system of FIG. 1, in accordance with aspects of the present disclosure.

FIG. 6 is a fragmentary side view of the ferrule and substitute wire of FIG. 1, with the ferrule pre-attached to the substitute wire.

FIG. 7 is another side view of the ferrule and pre-attached substitute wire of FIG. 6.

FIG. 8 is a side view of an exemplary dual-ferrule connector for attaching a broken wire to a substitute wire, in accordance with aspects of the present disclosure.

FIGS. 9 to 14 are exemplary configurations produced during performance of an exemplary method of replacing a broken wire with a substitute wire, taken generally along line 9-9 of FIG. 1 through the sternum, in accordance with aspects of the present disclosure.

FIG. 15 is a view of an adapter for transmitting compressive force from a hinged compression tool to a ferrule in order to crimp the ferrule, with the adapter operatively engaged with a ferrule that is illustrated in phantom outline, in accordance with aspects of the present disclosure.

FIG. 16 is a top view of the adapter and ferrule of FIG. 15, taken generally along line 16-16 of FIG. 15 before application of compressive force.

FIG. 17 is a top view of the adapter and ferrule of FIG. 15, taken generally as in FIG. 16 during application of compressive force by jaws of a compression tool shown in phantom outline.

FIG. 18 is a fragmentary side view of an exemplary holder engaged with and supporting the ferrule and the substitute wire of FIG. 1, in accordance with aspects of the present disclosure.

FIG. 19 is a top view of the holder, ferrule, and wire of FIG. 18, taken generally along line 19-19 of FIG. 18.

FIG. 20 is an end view of the holder, ferrule, and wire of FIG. 18, taken generally along line 20-20 of FIG. 18, with jaws of a compression tool illustrated in phantom outline and applying compressive force to the ferrule.

FIG. 21 is a fragmentary side view of another exemplary holder, with the holder engaged with and supporting the ferrule and the wires of FIG. 1, in accordance with aspects of the present disclosure.

FIG. 22 is an end view of the holder of FIG. 21, taken generally along line 22-22 of FIG. 21.

FIG. 23 is an end view of the holder of FIG. 21, taken generally along line 23-23 of FIG. 21.

FIG. 24 is a sectional end view of the holder of FIG. 21, taken generally along line 24-24 of FIG. 21, with jaws of a compression tool illustrated in phantom outline and applying compressive force to the ferrule.

### DETAILED DESCRIPTION

The present disclosure provides a system, including methods, apparatus, and kits, for replacing a damaged surgical wire, such as a surgical wire that has broken during installation as a suture (a fixation loop) for bone. The system may include a connector with at least one ferrule for attaching a substitute wire to a damaged wire, and provides a method of replacing a damaged wire with a substitute wire by using the damaged wire as a leader for travel of the substitute wire around bone.

The connector may include at least one ferrule forming at least one receptacle capable of receiving an end of a surgical wire, namely, a damaged wire, a substitute wire, or both interchangeably. The ferrule may be attached to the wire end, to secure the end of the wire to the ferrule. The ferrule may be attached by any suitable mechanism, such as deformation of the ferrule and/or wire, use of an adhesive, bonding, application of axial tension to the wire and ferrule (with a woven ferrule), or the like. For example, the ferrule may be deformable after receipt of the wire end, to secure the end of the wire to the ferrule. In some embodiments, the connector may include a pair of ferrules defining a pair of receptacles at opposing ends of the connector, to receive a wire end of the damaged wire and the substitute wire in respective receptacles. The pair of ferrules may be interconnected by an elongate spacer wire, which may be flexible to facilitate pulling the connector through soft tissue on a path around bone.

The system also may include an adapter configured to transmit compressive force to the connector, and particularly to a ferrule thereof, from a surgical tool (e.g., a needle driver, a wire cutter, a clamp with pivotable jaws, a combination thereof, etc.) that opposingly engages the adapter. The adapter may flex resiliently in response to compressive force applied by the surgical tool, which may urge arms of the adapter against at least generally opposing walls of the ferrule, to deform the ferrule against the wire. When the compressive force is removed, the arms of the adapter may (or may not) spring apart to permit removal of the adapter. In some embodiments, the arms of the adapter may be formed by discrete pieces, which may be connected to one another by a movable joint, such as a hinge joint.

The system further may include a holder to engage and support a ferrule as the ferrule is being attached to the end of one or more wires. The holder may include a proximal graspable handle and a distal mounting portion. The mounting portion may receive a ferrule placed axially or laterally (e.g., in a snap fit) into engagement with the mounting portion. The mounting portion may restrict axial motion of the ferrule in one or both axial directions defined by the ferrule, such that the ferrule does not slip as the ferrule is placed onto the end(s) of the wire(s). The holder may include at least one stop that overlaps a projecting portion of the mounted ferrule near one or both ends of the ferrule. The stop may be configured to be engaged by the jaws of a compression tool, as the tool is deforming the projecting portion of the mounted ferrule, to block complete closure of the jaws (and thus damage to the ferrule/wire).

The method may permit wire replacement during surgery, to rescue the installation of a damaged wire with a substitute wire. A damaged wire may be selected. The damaged wire may be a broken wire, which may have broken during its installation, such as while twisting together ends of the not-yet-broken wire. The damaged wire may extend around bone, with opposing ends of the wire accessible from the same general side of the bone. A substitute wire may be attached to the damaged wire using a connector including at least one ferrule. The connector may be attached to a prospective trailing end of the damaged wire and, optionally, to a prospective leading end of the substitute wire, or may be pre-attached to the leading end of the substitute wire, among others. In some examples, attachment of the connector to either or both wire ends may be achieved by deforming at least part of the connector (and/or either or both wire ends), such as by deforming at least one ferrule of the connector against either or both wire ends and/or twisting the ferrule onto either or both wire ends, among others. After attachment, the damaged wire may be pulled to urge the leading end of the substitute wire to travel behind the damaged wire and connector on a path extending through soft tissue and/or bone and around bone (i.e., a bone or bone portion). Once the leading end of the substitute wire becomes accessible by emerging from soft tissue and/or bone, the substitute wire may be advanced farther by pulling the damaged wire, the connector, the substitute wire, or a combination thereof. The substitute wire may be secured around bone after the substitute wire has taken the place of the broken wire around bone.

The wire replacement system disclosed herein has substantial advantages over other approaches to managing a damaged wire suture for bone. The advantages may include (1) replacement of only broken wire(s), (2) complete removal of a broken wire so that there is no residual broken wire of questionable strength and integrity, and/or (3) the substitute wire can be secured around bone in the same manner as other wire sutures that are not broken, among others.

These and other aspects of the present disclosure are described in the following sections: (I) overview of an exemplary wire replacement system, (II) exemplary connectors and substitute wires for wire replacement, (III) exemplary method of wire replacement, (IV) exemplary adapter for a compression tool, (V) exemplary holders for ferrules, and (VI) kits.

### I. Overview of an Exemplary Wire Replacement System

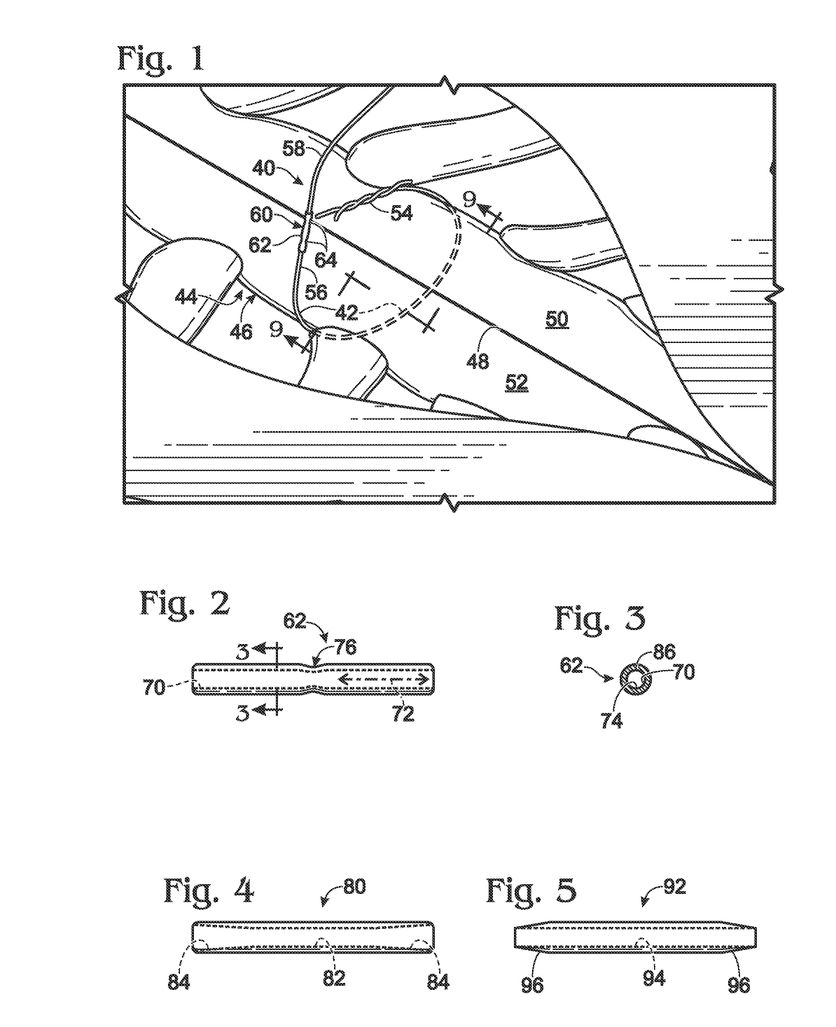

FIG. 1 shows selected aspects of a wire replacement system 40 attached to a broken wire 42 extending around bone 44, in this case, around a sternum 46. The sternum may have undergone a median sternotomy such that the sternum is split by a longitudinal cut 48 that forms left and right sternal fragments 50, 52. In other examples, the wire replacement system may be used to replace a damaged wire extending around a different bone and/or extending around a bone that has been fractured, among others.

Broken wire 42 may have a pair of ends 54, 56 disposed on the same general side of bone 44, and accessible to a surgeon. For example, here, wire 42 has a twisted end 54 and an untwisted end 56 created by breaking the wire at a site along a loop formed by the wire, such as breakage near a twisted-together portion of the wire.

Replacement system 40 may include a substitute wire 58, interchangeably termed a replacement wire, and a connector 60 that attaches the substitute wire to broken wire 42 at an end of the broken wire. The end of the broken wire may be created by breaking the wire and/or cutting the wire after breakage, among others. In the present illustration, substitute wire 58 is attached to untwisted end 56. In any event, the substitute wire may be a new wire that has not yet been twisted, kinked, or broken. Broken wire 42 may act as a leader that can be pulled to advance the substitute wire into position around sternum 46. Therefore, the substitute wire does not need to be attached to its own needle and can be advanced safely around the sternum without the need to open the chest cavity again.

The terms “wire” and “cable” in surgical applications are often used to distinguish respective monofilament and multi-stranded structures. Wires and cables thus may have distinct uses and properties (e.g., distinct flexibilities and tendencies to kink and fray). However, the term “wire,” as used in the present disclosure, is intended to encompass both monofilament and multi-stranded structures.

Connector 60 may include at least one ferrule 62, interchangeably termed a sleeve. In the depicted embodiment, the connector is composed of a single ferrule, which may be locked to both the broken wire and the substitute wire by any suitable mechanism(s), such as via crimps 64 introduced into the ferrule. In other cases, the connector may include at least a pair of ferrules (e.g., see Section II).

### II. Exemplary Connectors and Substitute Wires for Wire Replacement

This section describes exemplary connectors with one or more ferrules, and substitute wires for wire replacement; see FIGS. 2-8.

FIGS. 2 and 3 show respective side and sectional views of ferrule 62 before the ferrule has been attached to a wire. Ferrule 62 may be a tube and/or may have a tubular body extending along any suitable portion of the length of the ferrule, such as a majority or at least substantially all of the length of the ferrule.

The ferrule may define at least one bore 70 that extends along a long axis 72 of the ferrule, such as extending coaxially to the ferrule. The bore may be a through-bore that extends to each of the opposing ends of the ferrule, as shown here, or may be one or more blind bores that extend into but not through the ferrule from one or both opposing ends. The bore may be bounded by a smooth and/or featureless inner surface 74 of the ferrule. Alternatively, the inner surface may form projections or depressions, such as ridges or grooves, to facilitate engagement of the wire when the ferrule is deformed and/or when an end of the damaged wire is inserted into the ferrule. The bore and/or ferrule may (or may not) be cylindrical. In some cases, the bore may include a helical ridge or channel that engages the end of the damaged wire and encourages axial advancement as the ferrule is twisted onto (threaded onto) the end of the wire (and/or the end of the wire is twisted into (threaded into) the ferrule). The helical ridge or channel may deform the end of the wire as the wire and the ferrule are mated.

The bore may have any suitable diameter. The bore may (or may not) be slightly larger than the diameter of the wire(s) to be received, to facilitate placement of the wire into the bore, while allowing the wire to fit closely in the ferrule, such as before the ferrule is crimped. The bore may have a uniform diameter or may vary in diameter along its length. For example, the bore and/or ferrule may be pinched centrally along the ferrule, at a region indicated at 76 in FIG. 2, to narrow the bore in order to provide a barrier to wire travel that prevents over-insertion of a wire into the ferrule.

The ferrule may or may not be monolithically formed. In some cases, the ferrule may be a tubular, woven mesh formed of individual strands or bands. The mesh may function as a “Chinese finger trap” that contracts around and grips an end of a wire when the ferrule is tensioned axially.

FIG. 4 shows another exemplary ferrule 80 defining a bore 82. A broken wire may have a damaged end, which may be distorted and/or may have a burr or other projection. For this reason, bore 82 may flare toward one or both ends of the ferrule, indicated at 84, which may facilitate insertion of a damaged end of a wire into the ferrule. In some cases, the diameter of the bore may be different in respective approximate halves of the bore, to allow the ferrule to be used with a broken wire and a replacement wire of different respective diameters.

The ferrule may have any suitable (outside) diameter, which may be uniform or may vary along the length of the ferrule (see FIGS. 2 and 3). The diameter of the ferrule may be less than about twice and/or no more than about 50% greater than the diameter of the bore. Accordingly, the ferrule may have a wall 86 that is relatively thin, such as less than about one-half or one-fourth the diameter of the wire, to minimize the overall increase in diameter caused by the ferrule, thereby minimizing any step or other impediment to travel through soft tissue and/or bone that may be formed at the end of the ferrule. The ferrule also may (or may not) be rounded in profile at one or both ends, as shown in FIG. 2, to minimize damage to soft tissue that may be caused by travel of the ferrule.

FIG. 5 shows another exemplary ferrule 90 forming a wall 92 surrounding a bore 94. The thickness of wall 92 and/or the diameter of the ferrule may diminish toward one or both ends of the ferrule, indicated at 96, such that the ferrule tapers toward one or both ends. Wall 92 (and/or any other wall of a ferrule) may be circumferentially continuous or may form a longitudinal slot where the wall is absent.

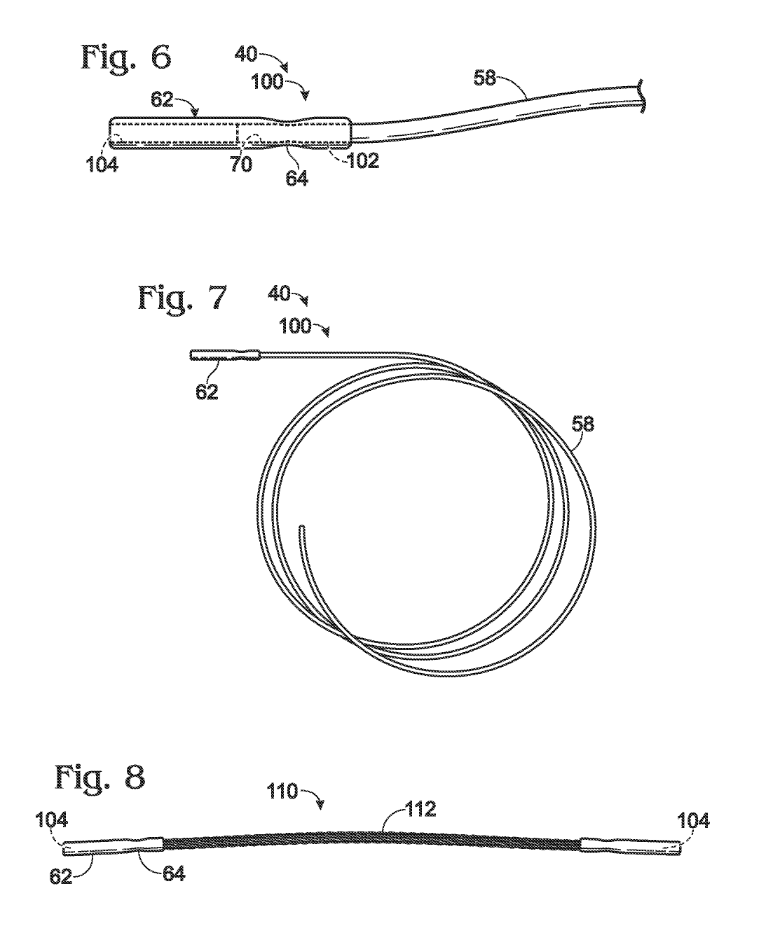

FIGS. 6 and 7 show a replacement assembly 100 of system 40 that includes ferrule 62 pre-attached to substitute wire 58. The ferrule may have any combination of the features and characteristics disclosed herein for other ferrules. The substitute wire may have any suitable length, but generally is substantially longer than the circumference of bone to be at least substantially encircled and fixed with the substitute wire.

An end 102 of substitute wire 58 may be disposed in bore 70. The ferrule may slide translationally onto the end of the substitute wire. Alternatively, the ferrule may be twisted onto the end of the wire, to attach the ferrule to the wire. In some cases, a crimp 64 may be formed in the ferrule (and substitute wire 58) to lock the substitute wire to the ferrule. Alternatively, or in addition, the substitute wire may be attached to the ferrule by any other suitable approach, such as soldering, welding, a press-fit, or the like. The ferrule may be attached during manufacture of the replacement assembly or by the surgeon, such as preoperatively or intraoperatively, among others. In any event, ferrule 62 may form a receptacle 104 for receiving broken wire 42. The receptacle may be described as a blind bore, which may be formed by the unoccupied portion of bore 70.

FIG. 8 shows an exemplary dual-ferrule connector 110 for attaching a broken wire to a substitute wire. The connector may (or may not) be pre-attached to a substitute wire during manufacture. Connector 110 may be equipped with a spacer wire 112 attached at opposing ends to a pair of ferrules 62. Each ferrule 62 may have any combination of the features and characteristics disclosed herein for ferrules and may be attached to spacer wire 112 by crimp 64 or by any other suitable approach. The ferrules of connector 110 may extend beyond the ends of spacer wire 112 to form a pair of receptacles 104 for receiving ends of wires. The ferrules may be substantial copies of each other, which may form receptacles that are about the same size, or the ferrules may be different in size or shape, such as to attach the opposing ends of connector 110 to wires of different diameter. Spacer wire 112 may (or may not) be more flexible than the broken wire and/or substitute wire, and thus may be a flexible member of monofilament (monolithic) or multi-filament construction.

The sleeves and wires disclosed herein may be formed of a biocompatible material(s). For example, the sleeves and/or wires may be formed of metal (e.g., stainless steel, which may be surgical grade stainless steel). A sleeve and each wire attached to the sleeve may be formed of the same material or respective different materials.

### III. Exemplary Method of Wire Replacement

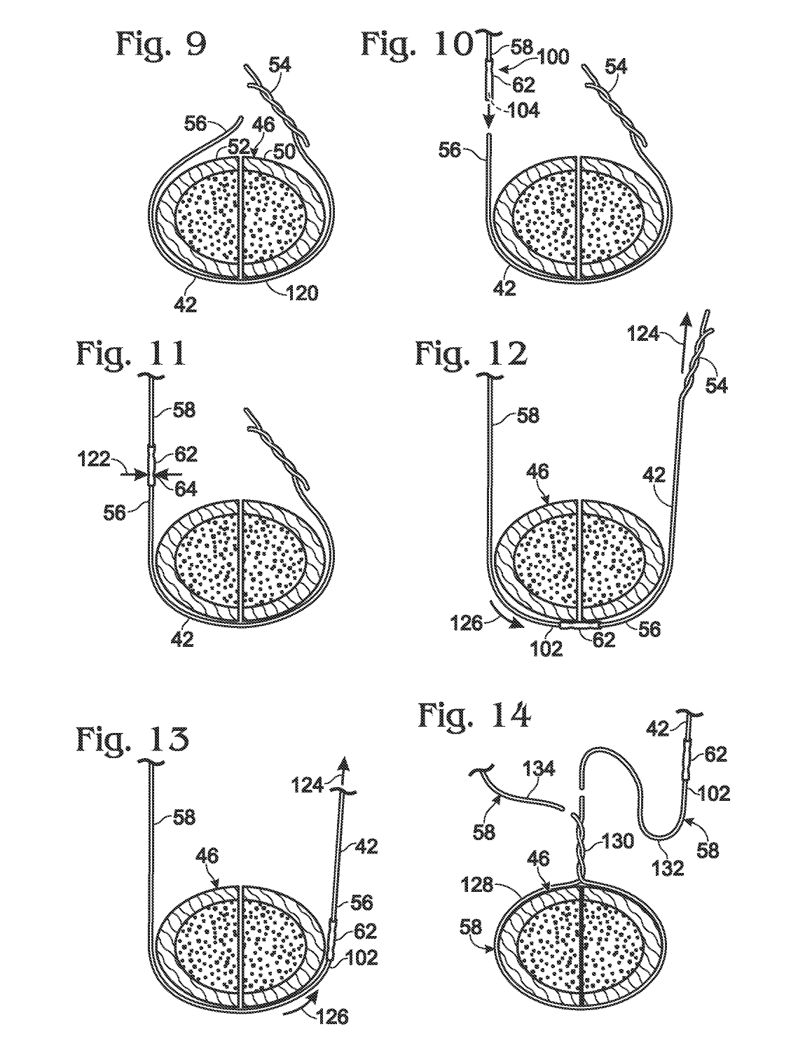

This section describes an exemplary method of replacing damaged surgical wire 42 with substitute wire 58; see FIGS. 9-14. The steps of the method may be performed in any suitable combination, in any suitable order, and using any combination of the system components of the present disclosure. The method is exemplified in the context of sternum 46 that has undergone a median sternotomy to produce sternal fragments 50, 52, but can be performed on any other suitable bone.

FIG. 9 shows broken wire 42 extending around sternum 46 immediately after breakage of the wire. Broken wire 42 may be selected for replacement. Generally, both ends 54, 56 of the broken wire are accessible to the surgeon, while an intervening region 120 is buried and extends under or through the bone. Accordingly, each end 54, 56 may be visible to the surgeon, may project from the same general side of the bone, and/or can be engaged readily by hand and/or with a tool. If one of the ends of the broken wire is buried under soft tissue and/or bone, the method may be more difficult to perform.

FIG. 10 shows replacement assembly 100 of wire replacement system 40 being moved into position for mating with untwisted end 56 of broken wire 42. More particularly, a connector including at least one ferrule, such as ferrule 62, may be placed onto wire end 56, such that the wire end is received in receptacle 104 of the ferrule. The ferrule already may be attached to substitute wire 58, or may be attached later.

The surgeon may select one of ends 54, 56 to serve as a leading end (in this case, end 54) for removal of broken wire 42, and the other end to function as a trailing end (in this case, end 56) as the broken wire is removed. This selection may, for example, be based on which end of broken wire 42 is the least twisted or kinked, which may be determined by where the break occurred in the wire. In some cases, the surgeon may prepare one of the broken wire ends to be received in receptacle 104 of the ferrule by cutting a short length from the wire end.

FIG. 11 shows ferrule 62 being attached to broken wire 42. Wire end 56 is disposed in ferrule 62 and the ferrule is being deformed by compression, indicated by arrows at 122, to form crimp 64. The ferrule may be deformed by application of compression with the jaws of a compression tool (e.g., pliers, a needle driver, a wire cutter, or the like), with the jaws engaged with the ferrule (e.g., see Section V). Alternatively, the ferrule may be deformed by application of compression with a compression tool and an adapter, with the jaws of the compression tool engaged with the adapter, and the adapter in turn engaged with the ferrule (e.g., see Section IV).The ferrule may be attached to the broken wire by any other suitable mechanism, such as an adhesive, bonding, soldering, twisting the ferrule onto the end of the wire, axial tension (with a woven ferrule that acts like a Chinese finger trap), or the like. In any event, broken wire 42 may be linked to substitute wire 58 with a connector that includes at least one ferrule. The ferrule may be engaged with each of the wires, or a pair of ferrules may be engaged with respective ends of the wires (e.g., see Section II).

FIGS. 12 and 13 shows broken wire 42 being pulled, indicated at 124, to advance, indicated at 126, substitute wire 58 around sternum 46. The broken wire may act as a leader that leads the ferrule and the substitute wire along a path around the sternum or other bone. The path may be defined by the broken wire acting as a leader for travel of the substitute wire. Stated differently, substitute wire 58 may be advanced around the sternum or other bone, with the substitute wire attached to the broken wire with at least one ferrule, and with the substitute wire trailing the broken wire as the broken wire is removed from the patient and from its position around bone. The substitute wire may be advanced until a leading end 102 of the substitute wire has emerged from its travel under the bone and extends far enough to allow a surgeon to manipulate the leading end directly.

FIG. 14 shows a portion 128 of substitute wire 58 secured around sternum 46. The ends of substitute wire portion 128 may be twisted together, indicated at 130, to secure the substitute wire adjacent the bone. Alternatively, the ends of substitute wire portion 128 may be engaged by a locking device, such as a locking sleeve and/or a crimp block, that is implanted with the substitute wire. Exemplary locking devices that may be suitable are described in U.S. Pat. No. 6,017,347 and U.S. Patent Application Publication No. 2010/0094294 A1, each of which is incorporated herein by reference. In any event, substitute wire portion 128 may at least substantially encircle bone when secured. Substitute wire portion 128 may be cut to disconnect the portion from broken wire 42, ferrule 62, and leading and trailing regions 132, 134 of substitute wire 58. Cutting may be performed at any suitable time, such as after leading end 102 of the substitute wire emerges from soft tissue and/or bone and becomes accessible to the surgeon.

### IV. Exemplary Adapter for a Compression Tool

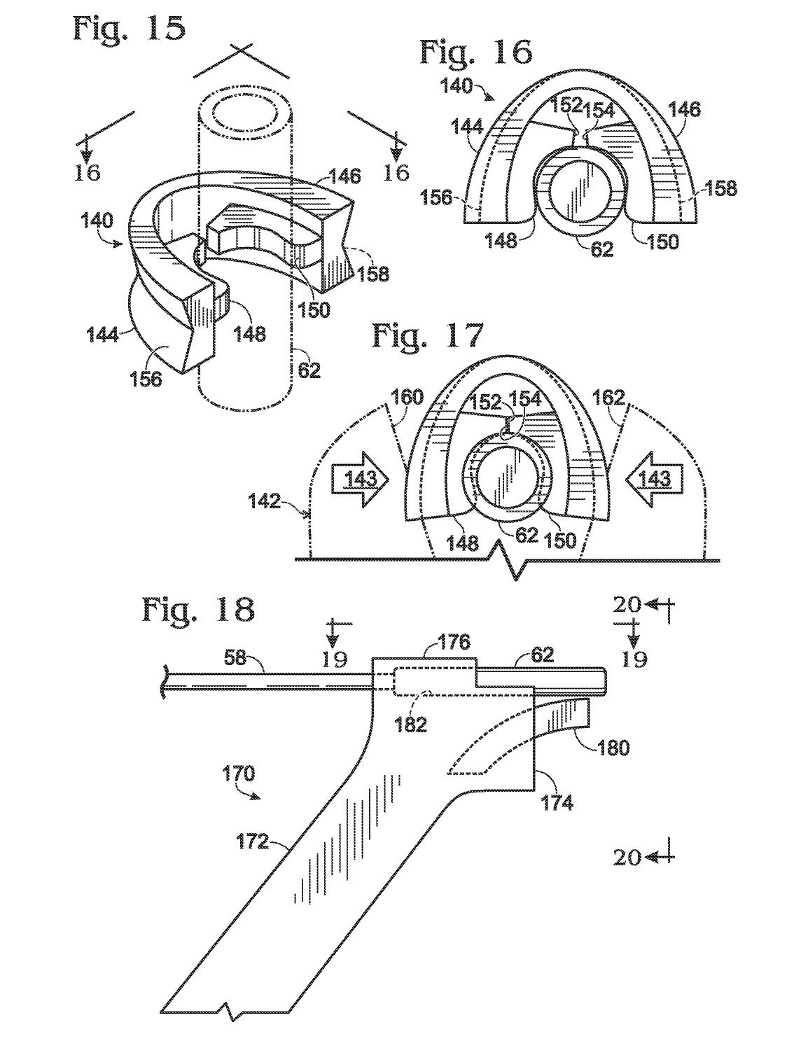

This section describes an exemplary adapter 140 in the form of a generally U-shaped clip that can be used in conjunction with a compression tool 142 to deform ferrule 62; see FIGS. 15-17.

The adapter may be configured as a removable accessory or attachment for use with compression tool 142, to transmit compressive force, indicated at 143 in FIG. 17, from the tool to the ferrule. The adapter may be resilient and may be formed as one piece or as at least two pieces that are pivotably connected by a hinge, among others. The adapter may form opposing arms 144, 146 that collectively embrace the ferrule, before and/or during application of compressive force. The arms may include at least one or a pair of opposing inner ridges 148, 150 at which compressive force is focused onto the ferrule. The arms also may be equipped with one or more stops 152, 154 that engage each other (compare FIGS. 16 and 17) when the ferrule is crimped sufficiently. Each arm may define an outer channel or recess 156, 158, which each may receive and be engaged by a respective jaw 160, 162 of the compression tool. Further aspects of adapters that may be suitable are described in U.S. Provisional Patent Application Ser. No. 61/409,415, filed Nov. 2, 2010, which is incorporated herein by reference.

### V. Exemplary Holders for Ferrules

This section describes exemplary holders configured to receive and support a ferrule, to facilitate mating and crimping the ferrule; see FIGS. 18-24.

FIG. 18 shows an exemplary holder 170 engaged with and supporting ferrule 62 and substitute wire 58. The holder may include a body 172 that forms a graspable handle, and a head 174 connected distally to the body. The head may provide a mounting portion or mount 176 to receive and hold the ferrule and the substitute wire. The head may be attached to and may support a stop 180 that limits movement of jaws of a compression tool toward each other.

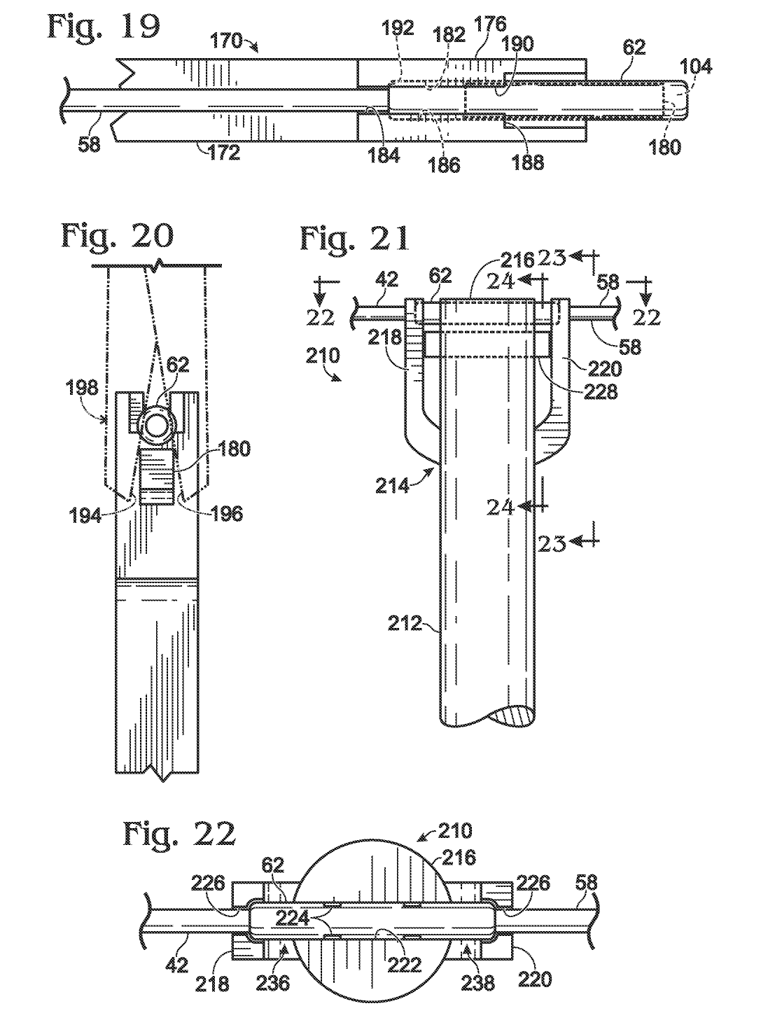

FIGS. 18 and 19 show aspects of mount 176. The mount may define a channel 182 sized to receive the wire and/or the ferrule. The channel may vary in width to provide a wire-receiving region 184 and a ferrule-receiving region 186. Channel 182 also may have a top wall portion 188 that extends over ferrule 62 and/or wire 58 to restrict removal of the wire and/or ferrule in a direction transverse to the long axis of the ferrule. In some cases, the top wall portion may form a gap 190 that permits the ferrule to be snap-fitted into mount 176 and then later removed from the mount through the gap. Alternatively, or in addition, the ferrule may be placed into and removed from the mount by axial motion of the ferrule (and wire 58) parallel to its long axis. In any event, mount 176 may provide a wall 192 that blocks axial motion of the ferrule toward substitute wire 58, to allow an axial load to be placed on the ferrule as the ferrule is being mated with an end of a broken wire.

Stop 180 may project from head 174 to overlap a region of the ferrule projecting from mount 176 (see FIGS. 18-20). The stop may be sized and positioned to limit how close jaws 194, 196 of a compression tool 198 can approach each other when the stop is disposed between the jaws and the jaws are engaged with (and crimping) the ferrule (see FIG. 20). For example, tool 198 may be a wire-cutting tool capable of cutting through the ferrule and the wire, if stop 180 is not present to block the jaws from closing completely. The stop may be formed of a hard material, such as hardened metal, to prevent the wire-cutting tool from cutting through the stop.

FIG. 21 shows another exemplary holder 210, with the holder engaged with and supporting ferrule 62 and wires 42, 58. Holder 210 may be utilized to hold ferrule 62 as each of the wires is received in and attached to the ferrule.

Holder 210 may include a body 212 forming a graspable handle, and a head 214 connected to the body. The head may form a mount 216 that receives ferrule 62. The head may include a pair of opposing arms 218, 220, each of which may be resilient, and which may engage opposing ends of ferrule 62 to restrict axial motion of the ferrule in both opposing directions. The arms thus may allow an axial load to be placed on the ferrule, without slippage of the ferrule, as the ferrule is being mated with (and attached to) each of the wires.

FIG. 22 shows a distal end view of holder 210. Mount 216 may define a channel 222 configured to receive ferrule 62 in a snap-fit connection. For example, the channel may be equipped with resilient tabs 224 that retain the ferrule after it is urged laterally into the channel, while permitting ferrule removal in the opposite direction after the wires have been attached to the ferrule.

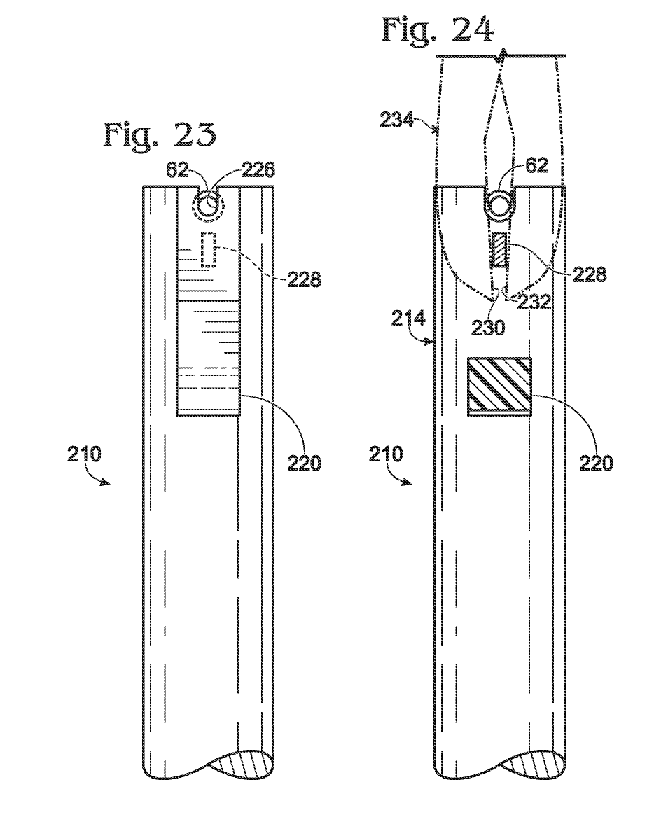

FIGS. 22 and 23 illustrate how arms 218, 220 restrict axial motion of ferrule 62. Each arm may define a notch 226 that is sized to receive the end of a wire but not the ferrule. As result, the arms block axial motion of the ferrule, because the ferrule cannot slide axially through either notch.

FIGS. 21 and 24 show a stop 228 supported by and projecting laterally from a mount 216 to positions adjacent each opposing end of ferrule 62. Stop 228 may be structured and may function generally as described above for stop 180 (see FIGS. 18-20). Stop 228 may be utilized to limit complete closure of jaws 230, 232 of a wire-cutting tool 234 when the jaws are positioned at each of two receiving sites 236, 238 flanking mount 216, namely, between mount 216 and each respective arm 218, 220 of the head (see FIG. 22).

### VI. Kits

The wire replacement system may be provided as a kit. The kit may include one or more connectors each including at least one ferrule, one or more substitute wires (optionally pre-attached to a ferrule), at least one wire pre-attached to a needle, one or more holders for a ferrule, at least one compression tool (e.g., a needle driver, a wire-cutting tool, forceps, a hemostat, or the like), one or more adapters/attachments for a compression tool, or any combination thereof.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure. Further, ordinal indicators, such as first, second, or third, for identified elements are used to distinguish between the elements, and do not indicate a particular position or order of such elements, unless otherwise specifically stated.

1. A method of surgical wire replacement, comprising:
selecting a damaged wire extending around bone;
attaching a substitute wire to an end of the damaged wire with a connector including at least one ferrule; and
pulling the damaged wire, with the damaged wire attached to the substitute wire, such that the substitute wire travels behind the damaged wire around the bone.

2. The method of claim 1, wherein the damaged wire is a broken wire extending around a cut sternum.

3. The method of claim 1, wherein the connector includes only one ferrule.

4. The method of claim 3, wherein the end of the damaged wire and an end of the substitute wire are both disposed in the only one ferrule after the step of attaching.

5. The method of claim 3, further comprising a step of disposing the end of the damaged wire in the ferrule before the step of attaching, wherein the ferrule is already attached to the substitute wire when the step of disposing is performed.

6. The method of claim 1, wherein the step of attaching includes a step of deforming a ferrule in which the end of the damaged wire is disposed.

7. The method of claim 6, wherein the step of deforming is performed with a wire-cutting tool engaged with the ferrule.

8. The method of claim 7, further comprising a step of connecting the ferrule to a holder that includes a stop, wherein the stop is positioned such that jaws of the wire-cutting tool engage the stop to prevent the jaws from closing completely.

9. The method of claim 8, wherein the step of connecting includes a step of mating the ferrule and the holder to create a snap-fit connection.

10. The method of claim 6, wherein the step of deforming is performed with a compression tool and an adapter, wherein the adapter is engaged opposingly with the ferrule, and wherein jaws of the compression tool are engaged opposingly with the adapter.

11. The method of claim 1, further comprising a step of securing at least a portion of the substitute wire adjacent the bone, with the portion of the substitute wire at least substantially encircling the bone.

12. The method of claim 11, wherein the step of securing includes a step of twisting together ends of the portion of the substitute wire.

13. A method of surgical wire replacement, comprising:
selecting a damaged wire extending around bone;
disposing a ferrule on an end of the damaged wire;
deforming the ferrule to attach the ferrule to the end of the damaged wire; and
advancing a substitute wire around the bone, with the damaged wire leading the substitute wire and attached to the substitute wire at least in part via the ferrule.

14. The method of claim 13, wherein the substitute wire is pre-attached to the ferrule.

15. The method of claim 13, wherein the step of advancing includes a step of pulling the damaged wire to urge the substitute wire around the bone.

16. The method of claim 13, wherein the step of deforming includes a step of engaging the ferrule with jaws of a compression tool.

17. The method of claim 13, further comprising a step of securing at least a portion of the substitute wire adjacent the bone, with the portion of the substitute wire at least substantially encircling the bone.

18. The method of claim 13, wherein the step of advancing is performed with the ferrule engaged with the damaged wire and the substitute wire.

19. A device for surgical wire replacement, comprising:
a surgical wire; and
a ferrule attached to an end of the surgical wire and forming a receptacle configured to receive an end of a damaged wire in coaxial relation with the end of the surgical wire.

20. The device of claim 19, wherein each of the surgical wire and the ferrule is formed of surgical grade stainless steel.
